(19) United States
(12) Patent Application Publication
(10) Pub. No.: US 2014/0088338 A1
(43) Pub. Date: Mar. 27, 2014

(54) CLOTHING WITH MAGNETS SYSTEMS

(71) Applicant: Alice Chang, South Pasadena, CA (US)

(72) Inventor: Alice Chang, South Pasadena, CA (US)

(21) Appl. No.: 13/769,761

(22) Filed: Feb. 18, 2013

Related U.S. Application Data
(60) Provisional application No. 61/705,710, filed on Sep. 26, 2012.

(51) Int. Cl. A61N 2/00

(52) U.S. Cl. CPC A61N 2/004; A61N 2/008
USPC 600/15

(57) ABSTRACT

Clothing with magnets systems provides a comfortable way to undergo magnetic therapy. The system covers a large surface area of the body with magnetic energy while enabling people to relieve pain, reduce inflammation, fight infection, support healing, reduce fluid retention, increase cellular oxygen, encourage deep restorative sleep, and a variety of other positive benefits. The system may be conveniently used for individuals who practice magnetic therapy.

### CROSS-REFERENCE TO RELATED APPLICATION

The present application is related to and claims priority from prior provisional application Ser. No. 61/705,710, filed Sep. 26, 2012 which application is incorporated herein by reference.

### COPYRIGHT NOTICE

A portion of the disclosure of this patent document contains material which is subject to copyright protection. The copyright owner has no objection to the facsimile reproduction by anyone of the patent document or the patent disclosure, as it appears in the Patent and Trademark Office patent file or records, but otherwise reserves all copyright rights whatsoever. 37 CFR 1.71(d).

### BACKGROUND OF THE INVENTION

The following includes information that may be useful in understanding the present invention(s). It is not an admission that any of the information provided herein is prior art, or material, to the presently described or claimed inventions, or that any publication or document that is specifically or implicitly referenced is prior art.

1. Field of the Invention

The present invention relates generally to the field of clothing and more specifically relates to clothing with magnets.

2. Description of the Related Art

Clothing is typically fiber and textile material worn as a covering on the body of a wearer. The wearing of clothing is mostly restricted to human beings and is a feature of nearly all modern human societies. The amount and type of clothing worn may depend on physical, social and geographic considerations. Clothing normally serves as a fashionable covering and as an insulator from ambient conditions; clothing is not traditionally multi-purpose in design.

A magnet is a material or object that produces a magnetic field. This magnetic field is invisible but is responsible for the most notable property of a magnet; a force that pulls on other ferromagnetic materials, such as iron, and attracts or repels other magnets. Magnets may serve many purposes. Magnetic therapy is an alternative medicine practice involving the use of static magnetic fields. Practitioners claim that subjecting certain parts of the body to magnetostatic fields produced by permanent magnets has beneficial health effects. Many people undergo magnetic therapy for a variety of different aliments and conditions. A convenient means by which magnetostatic fields can be introduced to the human body is desirable.

Various attempts have been made to solve the above-mentioned problems such as those found in U.S. Pat. and Pub. Nos. 5,720,046; 2005/0101828; 2007/0043254; 6,332,862; 3,943,912; and 2008/0051621. This art is representative of clothing. None of the above inventions and patents, taken either singly or in combination, is seen to describe the invention as claimed.

Ideally, a clothing with magnets should provide convenient magnetic therapy means and, yet would operate reliably and be manufactured at a modest expense. Thus, a need exists for a reliable clothing with magnets system to avoid the above-mentioned problems, one that may be worn with ease over a duration.

### BRIEF SUMMARY OF THE INVENTION

In view of the foregoing disadvantages inherent in the known clothing art, the present invention provides a novel clothing with magnets system. The general purpose of the present invention, which will be described subsequently in greater detail is to provide convenient magnetic therapy means for user-wearers.

The present invention provides people with an easy and comfortable way to undergo magnetic therapy. The clothing with magnets covers a large surface area of the body with magnetic energy while enabling people to relieve pain, reduce inflammation, fight infection, support healing, reduce fluid retention, increase cellular oxygen, encourage deep restorative sleep, and a variety of other things, yet not impeding them from completing their everyday tasks. The present invention is not cumbersome and may be ideal for individuals who practice magnetic therapy.

A clothing with magnets system is disclosed herein, in a preferred embodiment, comprising: a clothing garment assembly having an inner layer; an outer layer; a middle layer; and magnets. The inner layer, outer layer, and middle layer in combination form a garment for wear; wherein the magnets are interspersed in the middle layer (the middle layer located between the inner layer and the outer layer). The inner layer is such that it is located adjacent the body of a wearer when the garment is worn and the outer layer serving as protection from the elements. The middle layer may also comprise insulating means such as fill.

The magnets don't touch the wearer but are in relative proximity of the wearer when the garment is worn, the magnets thereby able to positively affect the body of the wearer. The magnets are embedded in the clothing garment assembly, with each North Pole of each magnet directly facing the body of the wearer for maximum benefit. In certain embodiments the magnets are directly sewn into the middle layer. Strength of the magnets is preferably 3950 Gauss, and other strengths can also be used. Embedded magnet may be disc-shaped 3950 Gauss ceramic North facing permanent magnets made of rubber. It should be noted that other Gauss levels may be used as per a desired strength of embedded magnet. The garment and magnets in combination, as such, form the clothing garment assembly; wherein the clothing with magnets system serves to relieve pain when used. In this way the magnets are useful to promote (positively influence) the health of the wearer.

A kit is also described herein including garments for comprising a clothing-line including: at least one shirt; at least one (pair of) pants; at least one item of nightwear (pajamas, robe or the like); at least one hat; at least one glove; at least one vest; at least one (set of leggings); at least one dress; at least one skirt; at least one (pair of) socks; at least one jacket; (or combination thereof) and at least one set of user instructions. The various items may be worn throughout the day and night.

A method of use for clothing with magnets is also disclosed herein preferably comprising the steps of: donning at least one garment, each of garment(s) comprising a clothing garment assembly having magnets; wearing clothing garment assembly to induce therapeutic treatment via magnets; and removing garment(s) after a wearer-defined duration.

The present invention holds significant improvements and serves as a clothing with magnets system. For purposes of summarizing the invention, certain aspects, advantages, and novel features of the invention have been described herein. It is to be understood that not necessarily all such advantages may be achieved in accordance with any one particular embodiment of the invention. Thus, the invention may be embodied or carried out in a manner that achieves or optimizes one advantage or group of advantages as taught herein without necessarily achieving other advantages as may be taught or suggested herein. The features of the invention which are believed to be novel are particularly pointed out and distinctly claimed in the concluding portion of the specification. These and other features, aspects, and advantages of the present invention will become better understood with reference to the following drawings and detailed description.

### BRIEF DESCRIPTION OF THE DRAWINGS

The figures which accompany the written portion of this specification illustrate embodiments and method(s) of use for the present invention, clothing with magnets, constructed and operative according to the teachings of the present invention.

FIG. 1 shows a perspective view illustrating clothing with magnets in an in-use condition according to an embodiment of the present invention.

FIG. 2 is a perspective view illustrating an item of clothing (shirt garment) of clothing with magnets according to an embodiment of the present invention of FIG. 1.

FIG. 3 is a perspective view illustrating item of clothing (pant garment) of clothing with magnets according to an embodiment of the present invention of FIG. 1.

FIG. 4A is a perspective view illustrating magnets of the clothing with magnets as used with a shirt according to an embodiment of the present invention of FIG. 1.

FIG. 4B is a perspective view illustrating magnets of the clothing with magnets as used with pants according to an embodiment of the present invention of FIG. 1.

FIG. 4C is a perspective view illustrating magnets of the clothing with magnets as used with a skirt and blouse (or dress) according to an embodiment of the present invention of FIG. 1.

FIG. 5 is a flowchart illustrating a method of use for clothing with magnets according to an embodiment of the present invention of FIGS. 1-4C.

The various embodiments of the present invention will hereinafter be described in conjunction with the appended drawings, wherein like designations denote like elements.

### DETAILED DESCRIPTION

As discussed above, embodiments of the present invention relate to a clothing device and more particularly to clothing with magnets as used to improve the conditions whereby magnetic therapy can be achieved over an extended duration in relative comfort.

Generally speaking, clothing with magnets of the present invention preferably provides people with the convenience of simply wearing clothing to undergo magnetic therapy. This innovative product may comprise a line of clothing with therapeutic magnets embedded or directly sewn into the clothing, with the magnet's North Pole directly facing the user's body. The line of clothing may include a variety of items, such as shirts, pants, pajamas, hats, face masks, gloves, t-shirts, vests, jackets, leggings, dresses, skirts, and socks. Other clothing items may also be included within the scope of the present invention. The clothing can be made from natural fabrics, such as cotton, linen, wool, down, and rubber, as well as synthetic fabrics, such as polyester and nylon and other suitable equivalents. The therapeutic magnets used may include ceramic magnets, neodymium magnets, samarium cobalt magnets, and flexible rubber magnets and other forms of magnets. Magnets may be embedded or directly sewn ‘or glued’ into the clothing. Outerwear may include jacket, rain jacket, down jacket, hooded jacket, windbreaker, and the like.

Types of Magnets that may be used for magnetic therapy as disclosed herein; Gauss readings vary within each type according to the magnet's weight, size and shape: Ceramic #5 and Ceramic #8 (Manufacturer's rating 3950 and 4000 Gauss) Actual readings with a Gauss meter will range from 300 to 1200 Gauss. Made by mixing ceramic clay and iron powder. Flexible magnetic rubber (Manufacturer's rating 700 Gauss) Actual readings with a Gauss meter will range from 120 to 260 Gauss. Made by mixing iron powder with rubber. Neodymium (Manufacturer's rating 10200 to 12300 Gauss). Actual readings with a Gauss meter will range from 1700 to 2300 Gauss. A very costly magnet made from a combination of neodymium, iron and boron. Samarium Cobalt (Manufacturer's rating 8700 to 1100 Gauss). Actual readings with a Gauss meter range from 1000 to 2000 Gauss. Made from a combination of samarium and cobalt. Magnet strength is measured in units called Gauss. For comparison, the natural magnetic field of the Earth is about ½ of 1 gauss. The average “refrigerator” magnet is about 10 gauss. Magnets used in therapeutic products may vary from about 100 gauss to 2,500 gauss. The strength of a magnet is dependent on the size, weight and shape of the magnet.

North-Negative Effects are used provides the following: has a counter-clockwise rotation; inhibits; relieves pain; reduces inflammation; produces an alkaline effect; reduces symptoms; rights infections; supports healing; reduces fluid retention; increases cellular oxygen; encourages deep restorative sleep; produces a bright mental effect; reduces fatty deposits; establishes healing polarity; stimulates melatonin production; and normalizes natural alkaline pH.

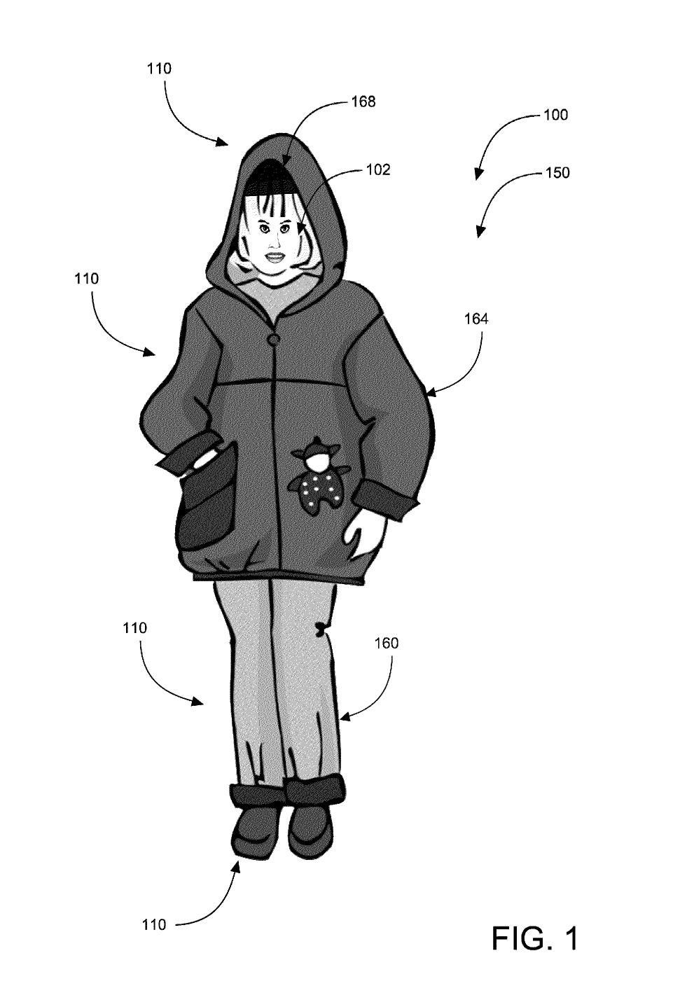

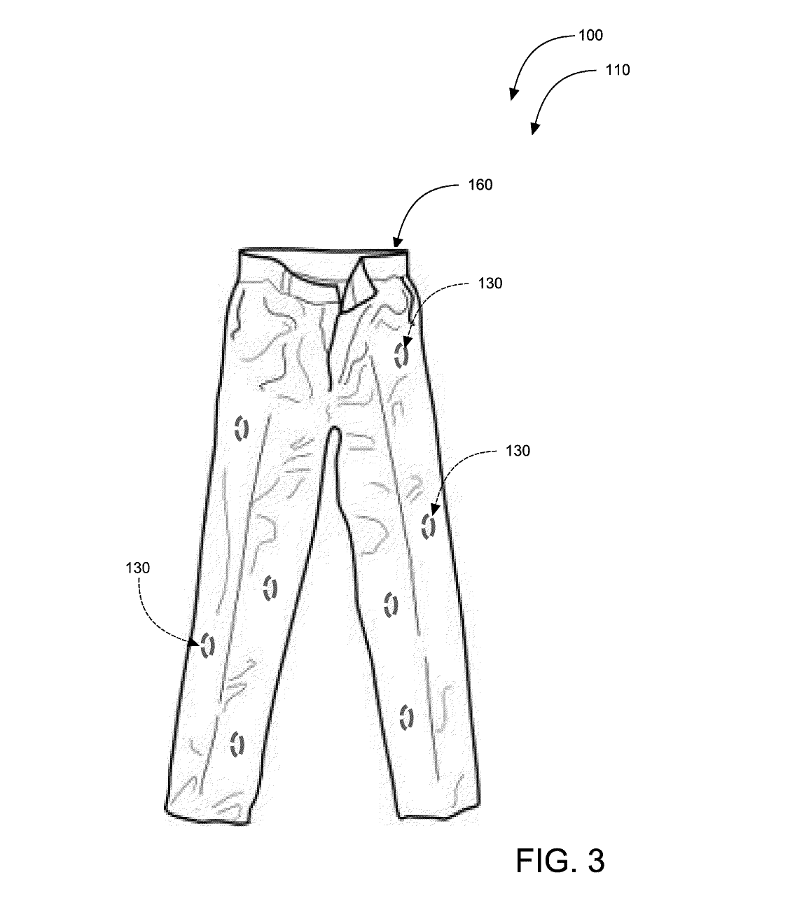

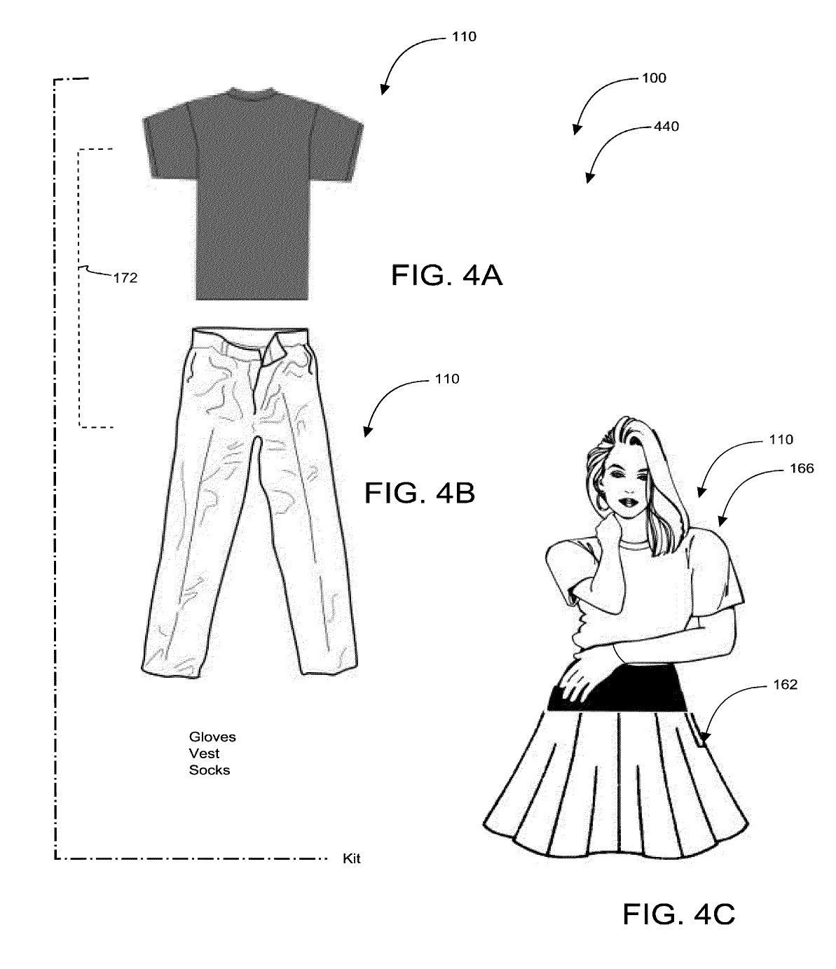

Referring now to the drawings more specifically by numerals of reference there is shown in FIGS. 1-4C perspective views illustrating clothing with magnets 100 in in-use condition 150 and various garments with magnets 130 that may be employed for use with the present invention.

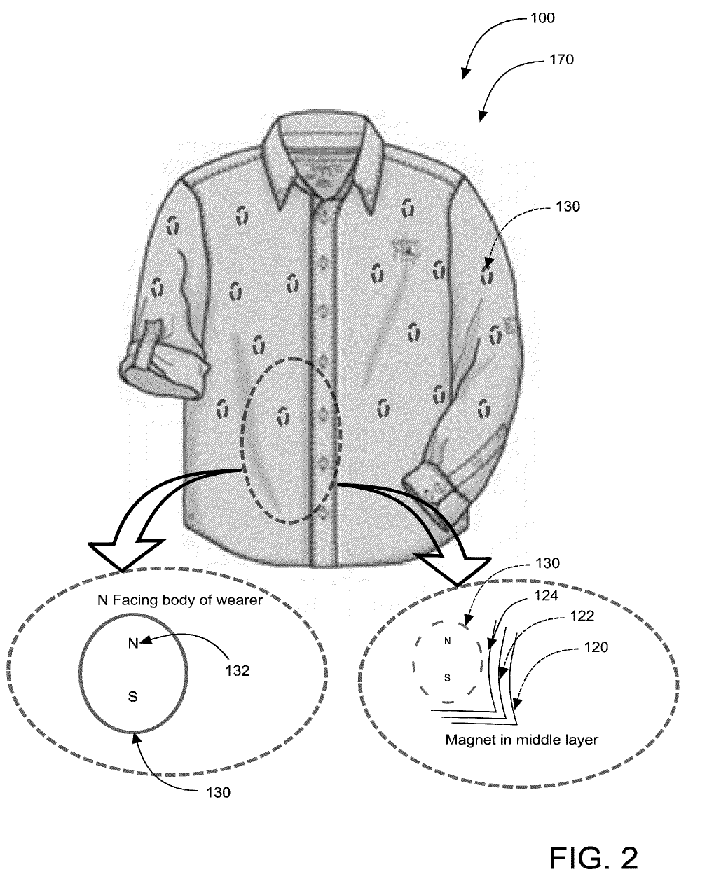

Clothing with magnets system 100 comprises at least one clothing garment assembly 110 having inner layer 120; middle layer 122; outer layer 124 (respectively from inside to outside of a garment); and magnets 130; wherein inner layer 120, outer layer 124, and middle layer 122 in combination form a garment for wear. More layers may be used if desired. Magnets 130 are preferably interspersed in middle layer 122; middle layer 122 located between inner layer 120 and outer layer 124; wherein inner layer 120 is located adjacent the body of wearer 102 when the garment is worn as indicated in FIG. 2.

Magnets 130 are held in relative proximity of wearer 102 when the garment is worn; magnets 130 thereby able to affect the body of wearer 102. Magnets 130 are preferably embedded in clothing garment assembly 110, with each North Pole 132 of magnets 130 directly facing the body of wearer 102, as mentioned previously and indicated in FIG. 2. In this way garment and magnets 130 in combination form clothing garment assembly 110; wherein magnets 130 are useful to promote the health of wearer 102 during in-use condition 150, as shown in FIG. 1.

As such, clothing with magnets system 100 serves to relieve pain for wearer 102 when used, to reduce inflammation, to fight infection to support healing, to reduce fluid retention, to increase cellular oxygen, and/or to encourage deep restorative sleep when used (such as when wearing nightwear 172).

Garment(s) may comprise pants 160; skirt 162; jacket 164; dress 166; hat 168; shirt 170; nightwear 172 (single piece, two-piece or multiple piece) and other clothing items. Magnets 130 may be directly sewn into middle layer in preferred embodiments as indicated in FIG. 2. Other fastening means may be used such as adhesive. Certain embodiments may use closable pockets or the like.

Magnets 130 of clothing with magnets system 100 are therapeutic magnets selected from the group consisting of ceramic magnets, neodymium magnets, samarium cobalt magnets, and flexible rubber magnets. Clothing may be selected from the group consisting of natural fabrics, such as cotton, linen, wool, down, and rubber, as well as synthetic fabrics, such as polyester and nylon. Other fabrics and materials may be used within the scope of the present invention.

Clothing with magnets system 100 may be sold as kit 440 including garments for comprising a clothing-line including: at least one shirt 170; at least one (pair of) pants 160; at least one item of nightwear 172 (pajamas or the like); at least one jacket 164; at least one hat 168; at least one (set of) gloves (not shown); at least one vest (not shown); at least one (set of legging-not shown); at least one dress 166; at least one skirt 162; at least one (pair of) socks (not shown); or combination thereof and at least one set of user instructions. Other garments may be included. Clothing with magnets system 100 may be manufactured and provided for sale in a wide variety of sizes and shapes for a wide assortment of applications. Upon reading this specification, it should be appreciated that, under appropriate circumstances, considering such issues as design preference, user preferences, marketing preferences, cost, structural requirements, available materials, technological advances, etc., other kit contents or arrangements such as, for example, including more or less components, customized parts, different color combinations, parts may be sold separately, etc., may be sufficient.

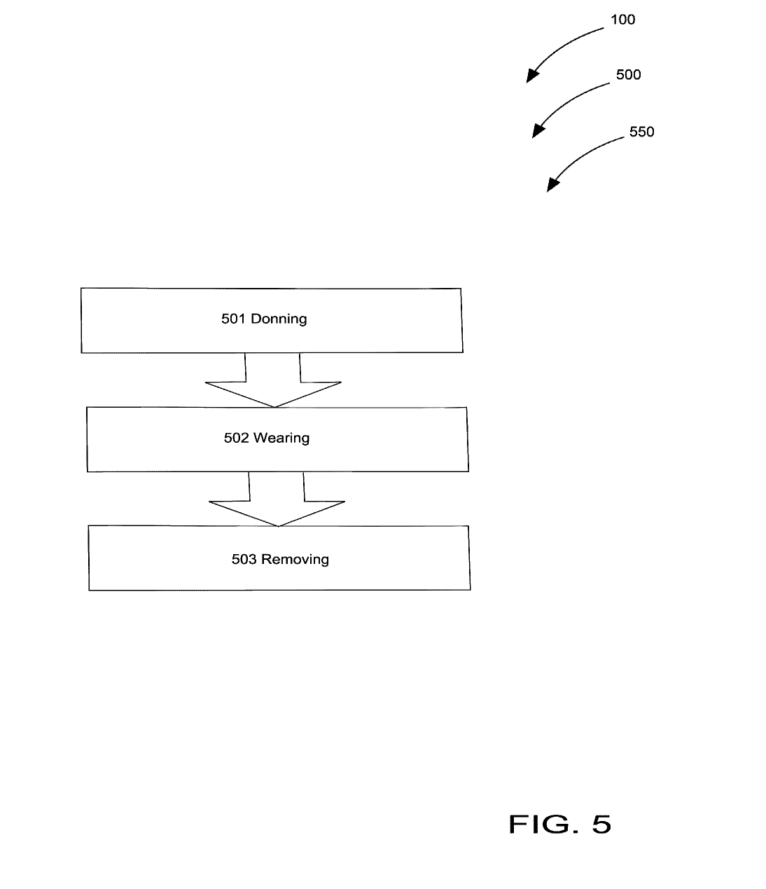

Referring now to FIG. 5, flowchart 550 illustrating a method of use 500 for clothing with magnets 100 according to an embodiment of the present invention of FIGS. 1-4C.

Method of using (method of use 500 for) clothing with magnets comprises the steps of: step one 501 donning at least one garment, each of the garment(s) comprising clothing garment assembly 110 having magnets 130; step two 502 wearing clothing garment assembly 110 to induce therapeutic treatment via magnets 130; and step three 503 removing the garment(s) after a wearer-defined duration.

It should be noted that the steps described in the method of use can be carried out in many different orders according to user preference. The use of “step of” should not be interpreted as “step for”, in the claims herein and is not intended to invoke the provisions of 35 U.S.C. §112, ¶6. Upon reading this specification, it should be appreciated that, under appropriate circumstances, considering such issues as design preference, user preferences, marketing preferences, cost, structural requirements, available materials, technological advances, etc., other methods of use arrangements such as, for example, different orders within above-mentioned list, elimination or addition of certain steps, including or excluding certain maintenance steps, etc., may be sufficient.

The embodiments of the invention described herein are exemplary and numerous modifications, variations and rearrangements can be readily envisioned to achieve substantially equivalent results, all of which are intended to be embraced within the spirit and scope of the invention. Further, the purpose of the foregoing abstract is to enable the U.S. Patent and Trademark Office and the public generally, and especially the scientist, engineers and practitioners in the art who are not familiar with patent or legal terms or phraseology, to determine quickly from a cursory inspection the nature and essence of the technical disclosure of the application.

What is claimed is new and desired to be protected by Letters Patent is set forth in the appended claims:

1. A clothing with magnets system comprising:
a clothing garment assembly having;
an inner layer;
an outer layer;
a middle layer; and
magnets;
wherein said inner layer, said outer layer, and said middle layer in combination form a garment for wear;
wherein said magnets are interspersed in said middle layer, said middle layer located between said inner layer and said outer layer;
wherein said inner layer is located adjacent a body of a wearer when said garment is worn;
wherein said magnets are in relative proximity of said wearer when said garment is worn, said magnets able to effect said body of said wearer;
wherein said magnets are embedded in said clothing garment assembly, with each north pole of said magnets directly facing said body of said wearer;
wherein said garment and said magnets in combination form said clothing garment assembly; and
wherein said magnets are useful to promote a health of said wearer.

2. The clothing with magnets system of claim 1 wherein said clothing with magnets system serves to relieve pain when used.

3. The clothing with magnets system of claim 2 wherein said clothing with magnets system serves to reduce inflammation when used.

4. The clothing with magnets system of claim 2 wherein said clothing with magnets system serves to fight infection when used.

5. The clothing with magnets system of claim 2 wherein said clothing with magnets system serves to support healing when used.

6. The clothing with magnets system of claim 2 wherein said clothing with magnets system serves to reduce fluid retention when used.

7. The clothing with magnets system of claim 2 wherein said clothing with magnets system serves to increase cellular oxygen when used.

8. The clothing with magnets system of claim 2 wherein said clothing with magnets system serves to encourage deep restorative sleep when used.

9. The clothing with magnets system of claim 2 wherein said garment comprises pants.

10. The clothing with magnets system of claim 2 wherein said garment comprises a skirt.

11. The clothing with magnets system of claim 2 wherein said garment comprises a jacket.

12. The clothing with magnets system of claim 2 wherein said garment comprises a dress.

13. The clothing with magnets system of claim 2 wherein said garment comprises a hat.

14. The clothing with magnets system of claim 2 wherein said garment comprises a shirt.

15. The clothing with magnets system of claim 8 wherein said garment comprises nightwear.

16. The clothing with magnets system of claim 2 wherein said magnets are directly sewn into said middle layer.

17. A clothing with magnets system comprising:
a clothing garment assembly having;
an inner layer;
an outer layer;
a middle layer; and
magnets;
wherein said inner layer, said outer layer, and said middle layer in combination form a garment for wear;
wherein said magnets are interspersed in said middle layer, said middle layer located between said inner layer and said outer layer;
wherein said inner layer is located adjacent a body of a wearer when said garment is worn;
wherein said magnets are in relative proximity of said wearer when said garment is worn, said magnets able to effect said body of said wearer;
wherein said magnets are embedded in said clothing garment assembly, with each north pole of said magnets directly facing said body of said wearer;
wherein said magnets are directly sewn into said middle layer;
wherein said garment and said magnets in combination form said clothing garment assembly;
wherein said clothing with magnets system serves to relieve pain when used; and
wherein said magnets are useful to promote a health of said wearer.

18. The clothing with magnets system of claim 17 further comprising a kit including said garments for comprising a clothing-line including:
said shirt(s);
said pant(s);
said nightwear;
said hat(s);
face mask(s);
glove(s);
vest(s);
said jacket(s);
legging(s);
said dress(es);
said skirts;
socks; and
a set of instructions for use.

19. The clothing with magnets system of claim 17 wherein said magnets are therapeutic said magnets selected from the group consisting of ceramic said magnets, neodymium said magnets, samarium cobalt said magnets, and flexible rubber said magnets

20. A method of using clothing with magnets comprising the steps of:
donning at least one garment, each of said garment(s) comprising clothing garment assembly having magnets;
wearing said clothing garment assembly to induce therapeutic treatment via said magnets; and
removing said garment(s) after a wearer-defined duration.
